(19) United States
(12) Patent Application Publication
(10) Pub. No.: US 2022/0164094 A1
(43) Pub. Date: May 26, 2022

(54) SYSTEMS AND METHODS FOR ADJUSTING A SETTING USING A USER INTERFACE

(71) Applicant: GE Precision Healthcare LLC, Wauwatosa, WI (US)

(72) Inventor: Eero Tapio Hyytiäinen, Helsinki (FI)

(21) Appl. No.: 17/104,726

(22) Filed: Nov. 25, 2020

(51) Int. Cl. G06F 3/0484; G06F 3/0482; G06F 3/0488; G16H 40/63

(52) U.S. Cl. CPC G06F 3/04847; G16H 40/63; G06F 3/04883; G06F 3/0482

(57) ABSTRACT

Embodiments for adjusting a setting are described herein. In some examples, a system can include a processor that can detect a setting selection with a user interface displayed with a touchscreen display device and generate a modified setting menu to be displayed with the user interface, wherein the modified setting menu comprises at least an adjusted setting value and a cancel indicator, wherein the cancel indicator provides a current setting value. The processor can also determine whether a confirmation selection confirms the adjusted setting value and, if so, modify the setting based on the confirmation selection. The processor can also determine whether a cancellation selection of the cancel indicator is detected and, if so, close the modified setting menu without adjusting the setting.

### FIELD

Embodiments of the subject matter disclosed herein relate to adjusting a setting using at least a user interface provided by a touchscreen display.

### BACKGROUND

Various electronic devices can display information using any number of display devices. In some examples, the display devices can include functionality for receiving or otherwise obtaining input using a touchscreen display. For example, a user interface can be displayed by the touchscreen display and a user can provide user input by contacting the touchscreen display. The user interface can detect the location of contact by the user and perform a set of instructions such as storing user input, causing a new application to be executed, or the like.

### SUMMARY

This summary introduces concepts that are described in more detail in the detailed description. It should not be used to identify essential features of the claimed subject matter, nor to limit the scope of the claimed subject matter.

In some aspects, a system can include a processor that can detect a setting selection with a user interface displayed with a touchscreen display device and generate a modified setting menu to be displayed with the user interface, wherein the modified setting menu comprises at least an adjusted setting value and a cancel indicator, and wherein the cancel indicator provides a current setting value. The processor can also determine whether a confirmation selection confirms the adjusted setting value and, if so, modify the setting based on the confirmation selection. The processor can also determine whether a cancellation selection of the cancel indicator is detected and, if so, close the modified setting menu without adjusting the setting.

In some examples, the system can be a respiratory device or an anesthesia device. The setting can include an alarm setting, a respiratory parameter, or a gas control parameter. The gas control parameter can include a gas flow, NO2 flow, an oxygen flow, an air flow, a total flow, an anesthetic agent percentage, a carbon dioxide setting, or an oxygen percentage. In some examples, the respiratory parameter can include a tidal volume, a ventilation pressure, or a respiration rate.

In some examples, the modified setting menu can also include an adjustment widget that detects an increased adjustment to the setting or a decreased adjustment to the setting. In some examples, the processor can modify the adjusted setting value in response to an adjustment selection of the adjustment widget, wherein the adjustment widget is a spinner widget. The determining whether the confirmation selection confirms the adjusted setting value can include, in some examples, detecting a confirmation gesture applied to the user interface. The confirmation gesture can include a force applied to a region of the user interface that displays the adjusted setting value, wherein the force is applied for a period of time that exceeds a threshold. In some examples, the confirmation gesture can include a force applied to a region of the user interface that displays the adjusted setting value followed by a release of the force or wherein the confirmation gesture comprises a pushing force applied to a control knob of the system followed by a release of the force applied to the control knob. In some examples, the confirmation gesture can include a force applied to a region of the user interface that excludes both a display of the adjusted setting value and a dead space region proximate the display of the adjusted setting value.

In some aspects, the modified setting menu can include one or more predetermined values for the setting and the processor can determine the one or more predetermined values for the setting based on historical physiological data for a patient. In some examples, the processor can modify the adjusted setting value in response to a control knob input detected by a control knob electronically coupled to the system.

In some aspects, a method for controlling healthcare settings can include detecting a setting selection with a user interface displayed with a touchscreen display device and generating a modified setting menu to be displayed with the user interface, wherein the modified setting menu comprises at least an adjusted setting value and a cancel indicator, wherein the cancel indicator provides a current setting value. In some examples, the method can include determining whether a confirmation selection confirms the adjusted setting value and, if so, modifying the setting based on the confirmation selection and determining whether a cancellation selection of the cancel indicator is detected and, if so, closing the modified setting menu without adjusting the setting.

In some examples, a non-transitory computer-readable medium can include a plurality of machine-executable instructions that, in response to execution by a processor, cause the processor to detect a setting selection with a user interface displayed with a touchscreen display device and generate a modified setting menu to be displayed with the user interface, wherein the modified setting menu comprises at least an adjusted setting value and a cancel indicator, wherein the cancel indicator provides a current setting value. In some examples, the plurality of instructions can cause the processor to determine whether a confirmation selection confirms the adjusted setting value and, if so, modify the setting based on the confirmation selection. In some examples, the plurality of instructions can cause the processor to determine whether a cancellation selection of the cancel indicator is detected and, if so, close the modified setting menu without adjusting the setting.

It should be understood that the brief description above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

The present examples will be better understood from reading the following description of non-limiting embodiments, with reference to the attached drawings, wherein below:

FIG. 1 schematically shows a system for modifying a setting of an electronic device, according to examples herein.

FIG. 2 shows an example process flow diagram of a modifying a setting of an electronic device using a modified setting menu, according to examples herein.

FIG. 3A is an example user interface with a modified setting menu, according to examples herein.

FIG. 3B provides an example modified setting menu, according to examples herein.

FIG. 4 shows an example block diagram of confirmation gestures detectable by the user interface providing the modified setting menu, according to examples herein.

FIG. 5 shows an example respiratory or anesthesia device with a control knob and a touchscreen display according to examples herein.

FIG. 6 shows an example non-transitory computer-readable medium for modifying a setting using a modified setting menu, according to examples herein.

### DETAILED DESCRIPTION

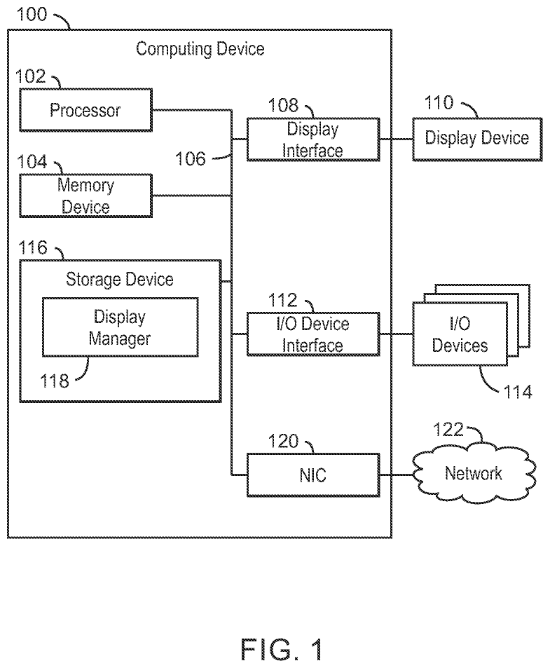

Embodiments of the present disclosure will now be described, by way of example, with reference to FIGS. 1-6, which relate to various embodiments of a system that facilitates adjusting a setting for the system using at least a user interface and a touchscreen display. In some examples, the system can also receive or otherwise obtain input from a control knob, among other components, wherein the input can be used to modify the setting. The user interface described herein can be modified to include a modified setting menu that can change any suitable setting value displayed or otherwise provided by the user interface.

In some examples, the modified setting menu can be activated in response to a selection of a value to be adjusted or modified. For example, a user interface can display any number of values that can be selected and in response to a selection, a modified setting menu can appear overlaying the displayed value. In some examples, the modified setting menu can provide a display of an adjusted value, any number of icons, widgets, and the like, that enable increasing or decreasing the setting value, and a cancel indicator that returns to the user interface without adjusting the value.

The technical effect of providing a modified setting menu enables a setting value provided by a user interface to be modified without displaying menus that overlap or otherwise obstruct additional values being displayed. The unobstructed values may include data, such as patient information, and the like, determined to have a rank or importance level above a predetermined threshold. In some examples, the modified setting menu can enable modifying setting values while covering or obstructing less data than current known techniques. The present techniques have a technical advantage of providing a modified setting menu that enables the adjustment of a setting value without obstructing the view of additional displayed values or with less obstruction of additional displayed values than conventional user interfaces. In some examples, the techniques herein can also include functionality for confirming or canceling an adjustment to a setting value. The present techniques can prevent an electronic device from inadvertently hiding critical display data in health care devices, industrial devices, or the like. The present techniques can also ensure that an electronic device is capable of adjusting or cancelling the adjustment of a setting value while enabling the user interface to provide other information without resizing or otherwise manipulating the user interface.

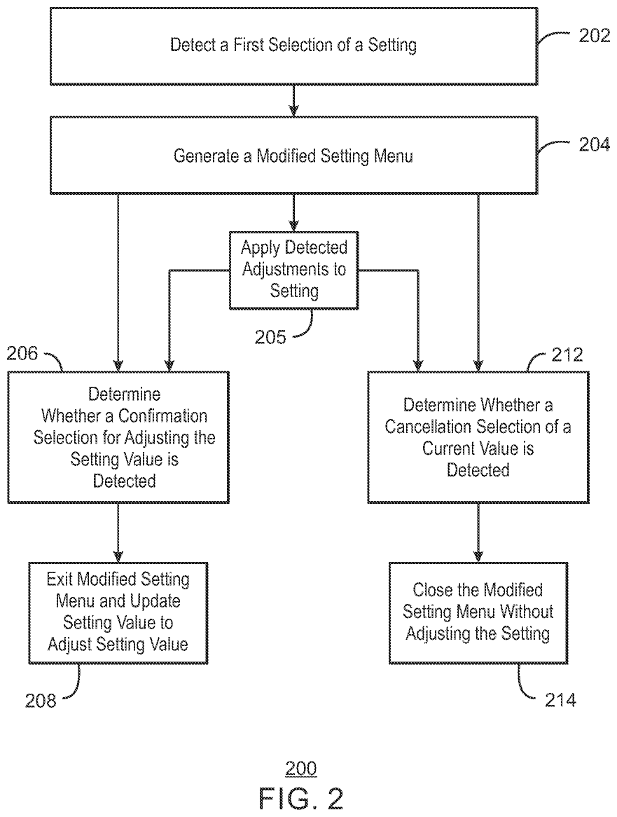

FIG. 1 is a block diagram of an example of a computing device that can adjust a setting of an electronic device. The computing device 100 may be, for example, a hospital monitor, an anesthesia device, an ICU ventilator, a respiratory device, an imaging device, a laptop computer, a desktop computer, a tablet computer, or a mobile phone, among others. The computing device 100 may include a processor 102 that is adapted to execute stored instructions, as well as a memory device 104 that stores instructions that are executable by the processor 102. The processor 102 can be a single core processor, a multi-core processor, a computing cluster, or any number of other configurations. The memory device 104 can include random access memory, read only memory, flash memory, or any other suitable memory systems. The instructions that are executed by the processor 102 may be used to implement a method that can adjust a setting of a device, as described in greater detail below in relation to FIG. 2. The processor 102 may also be linked through the system interconnect 106 (e.g., PCI, PCI-Express, NuBus, etc.) to a display interface 108 adapted to connect the computing device 100 to a display device 110. The display device 110 may include a display screen that is a built-in component of the computing device 100. The display device 110 may also include a computer monitor, television, or projector, among others, that is externally connected to the computing device 100. The display device 110 can include light emitting diodes (LEDs), and micro-LEDs, Organic light emitting diode OLED displays, among others.

The processor 102 may be connected through a system interconnect 106 to an input/output (I/O) device interface 112 adapted to connect the computing device 100 to one or more I/O devices 114 The I/O devices 114 may include, for example, a keyboard and a pointing device, wherein the pointing device may include a touchpad or a touchscreen, among others. The I/O devices 114 may be built-in components of the computing device 100 or may be devices that are externally connected to the computing device 100.

In some embodiments, the processor 102 may also be linked through the system interconnect 106 to a storage device 116 that can include a hard drive, an optical drive, a USB flash drive, an array of drives, or any combinations thereof. In some embodiments, the storage device 116 can include any suitable applications. In some embodiments, the storage device 116 can include a display manager 118, among others applications. In some embodiments, the display manager 118 can detect a setting selection with a user interface displayed with a touchscreen display device. In some examples, the setting can include a respiratory rate, an anesthetic agent percentage, a carbon dioxide percentage, or a tidal volume, among others. The display manager 118 can also generate a modified setting menu to be displayed with the user interface, wherein the modified setting menu comprises at least an adjusted setting value and a cancel indicator. The cancel indicator, as referred to herein, can include any suitable icon, widget, user interface input region, or the like, that can close the modified setting menu without adjusting or otherwise modifying a setting value. The cancel indicator can also provide a representation of the current setting value.

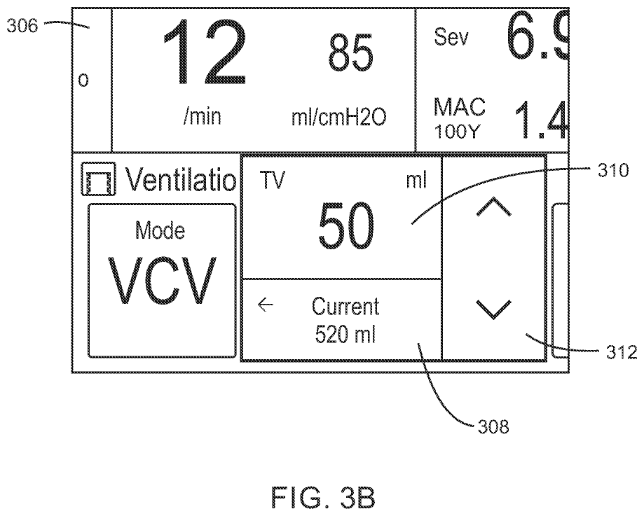

In some examples, the display manager 118 can also determine whether a confirmation selection confirms the adjusted setting value and, if so, modify the setting based on the confirmation selection. Determining whether the selection confirms the adjusted setting value can include detecting a confirmation gesture applied to the user interface. The confirmation gestures are described in greater detail below in relation to FIG. 4. In some examples, the display manager 118 can also determine whether a selection of the cancel indicator is detected and, if so, close the modified setting menu without adjusting the setting.

In some examples, a network interface controller (also referred to herein as a NIC) 120 may be adapted to connect the computing device 100 through the system interconnect 106 to a network 122. The network 122 may be a cellular network, a radio network, a wide area network (WAN), a local area network (LAN), or the Internet, among others. The network 122 can enable data, such as alerts, among other data, to be transmitted from the computing device 100 to remote computing devices, remote display devices, and the like. In some examples, the NIC 120 can enable controlling the settings of the computing device 100 using a remote system accessible by the network 122 For example, the NIC 120 may provide a remote system with a setting selection from the computing device 100 that causes the generation of the modified setting menu. In some examples, the NIC 120 can provide the modified setting menu to the remote system for display and an adjustment to a setting can be obtained from the remote system by the NIC 120. The functionality of the confirmation selection and the cancellation selection can also be provided to the remote system along with the modified setting menu.

It is to be understood that the block diagram of FIG. 1 is not intended to indicate that the computing device 100 is to include all of the components shown in FIG. 1. Rather, the computing device 100 can include fewer or additional components not illustrated in FIG. 1 (e.g., additional memory components, embedded controllers, additional modules, additional network interfaces, etc.). Furthermore, any of the functionalities of the display manager 118 may be partially, or entirely, implemented in hardware and/or in the processor 102. For example, the functionality may be implemented with an application specific integrated circuit, logic implemented in an embedded controller, or in logic implemented in the processor 102, among others. In some embodiments, the functionalities of the display manager 118 can be implemented with logic, wherein the logic, as referred to herein, can include any suitable hardware (e.g., a processor, among others), software (e.g., an application, among others), firmware, or any suitable combination of hardware, software, and firmware.

FIG. 2 illustrates a process flow diagram of an example method for adjusting a setting of an electronic device using a modified setting menu. In some examples, the method 200 can be implemented with any suitable computing system, such as the computing device 100 of FIG. 1, among others.

At block 202, the method 200 can include detecting a setting selection with a user interface displayed with a touchscreen display device. In some examples, the setting selection can be detected by a user selection of a region of a display device that is representing the setting. The user selection can be detected, received, or otherwise obtained from a mouse, keyboard input, pointing device input, a stylus device, a remote device, or contact with a touchscreen display, among others. The setting can represent any suitable value such as physiological data from a patient, breathing gas data, or the like. In some examples, the setting can include an alarm setting, a respiratory parameter, or a gas control parameter. The gas control parameter can include a gas flow, NO2 flow, an oxygen flow, an air flow, a total flow, an anesthetic agent percentage, a carbon dioxide setting, or an oxygen percentage, among others. The respiratory parameter can include a tidal volume, a ventilation pressure, or a respiration rate, among others. In some examples, the alarm setting can include a low threshold or a high threshold associated with a respiratory parameter or a gas control parameter.

At block 204, the method 200 can include generating a modified setting menu to be displayed with the user interface. In some examples, the modified setting menu is generated in response to the selection of the setting being modified. The modified setting menu can be displayed in the region of a user interface that displays the value for the setting being modified, adjacent to the displayed setting being modified, or within any suitable region of a user interface. In some examples, the modified setting menu can be sized to prevent the modified setting menu from blocking information or data displayed within the user interface. For example, the modified setting menu can be generated in a smaller representation or a larger representation depending on a size of the region of the user interface displaying the setting value. The modified setting menu can also be generated in varying sizes based on a size of the display device providing the user interface.

The modified setting menu, as referred to herein, can include at least an adjusted setting value and a cancel indicator, among others. The modified setting menu can also include, in some examples, an icon, widget, spinner, or the like, that can enable increasing, decreasing, or otherwise modifying the setting that is being adjusted. In some examples, the adjusted setting value can represent the modified setting value and a region of the user interface displaying the adjusted setting value can be swiped to increase, decrease, or modify the adjusted setting value. In some examples, the modified setting menu can include additional icons, widgets, and the like, such as predetermined value icons representing suggested adjusted setting values, a combination of settings to be adjusted, or the like. In some examples, the cancel indicator, as referred to herein, of the modified setting menu can include any icon, widget, or region of a display device that, when selected, prevents any adjustments from being applied to a selected setting or value. The cancel indicator can provide or display a current setting value. This enables a user to view the current setting value as the modified setting menu is displayed or provided in the space or region of the user interface that previously provided the current setting value. In some examples, the cancel indicator can also include any suitable information, such as a title indicating the value provided by the cancel indicator is the current setting value, or the like. In some examples, the modified setting menu can include providing an information dialog box proximate to the modified setting menu, or any other additional information. The information dialog box can include troubleshooting data, and the like.

At block 205, the method 200 can apply one or more detected adjustments to the setting. For example, the method 200 can include detecting one or more adjustments to the setting provided or otherwise obtained from a control knob, an adjustment widget, a remote system, or the like.

At block 206, the method 200 can include determining whether a confirmation selection confirms the adjusted setting value and, if so, at block 208, the method can include exiting the modified setting menu and updating the setting based on the adjusted setting value. In some examples, the adjusted setting value can be the same as the setting value, so that no updates are executed. In some examples, the adjusted setting value can be confirmed with any suitable confirmation gesture applied to the modified setting menu, an area of the user interface outside of the modified setting menu, or an area outside of a dead space proximate to the modified setting menu. The confirmation gesture can be a point and hold gesture that exceeds a predetermined amount of time, a swipe in any number of predetermined directions, a sequence of swipes in any number of predetermined directions, or the like. In some examples, the modified setting menu can detect adjustments to the setting value through input from the adjustment widget, scrolling of the displayed adjustment setting itself, or using any other suitable technique.

At block 212, the method 200 can include determining or detecting whether a cancellation selection of a cancel indicator displaying a current value button, widget, or region of a user interface is detected. If so, the method can include, at block 214, closing the modified setting menu without adjusting the setting. For example, selection of the cancel indicator can result in the modified setting menu closing and returning to the user interface in which the setting value is displayed. In some examples, areas of the user interface outside of the modified setting menu may not accept input or any selection when the modified setting menu is displayed. The cancel indicator can return the user interface to full functionality that includes the selection of any suitable icons, values, widgets, and the like.

The process flow diagram of method 200 of FIG. 2 is not intended to indicate that all of the operations of blocks 202-214 of the method 200 are to be included in every example. Additionally, the process flow diagram of method 200 of FIG. 2 describes a possible order of executing operations. However, it is to be understood that the operations of the method 200 can be implemented in various orders or sequences. In addition, in some examples, the method 200 can also include fewer or additional operations. For example, the method 200 can include modifying the adjusted setting value in response to an adjustment selection of the adjustment widget, wherein the adjustment widget is a spinner widget. A widget, as referred to herein, can include any suitable user interface feature that enables the selection of a region of a user interface.

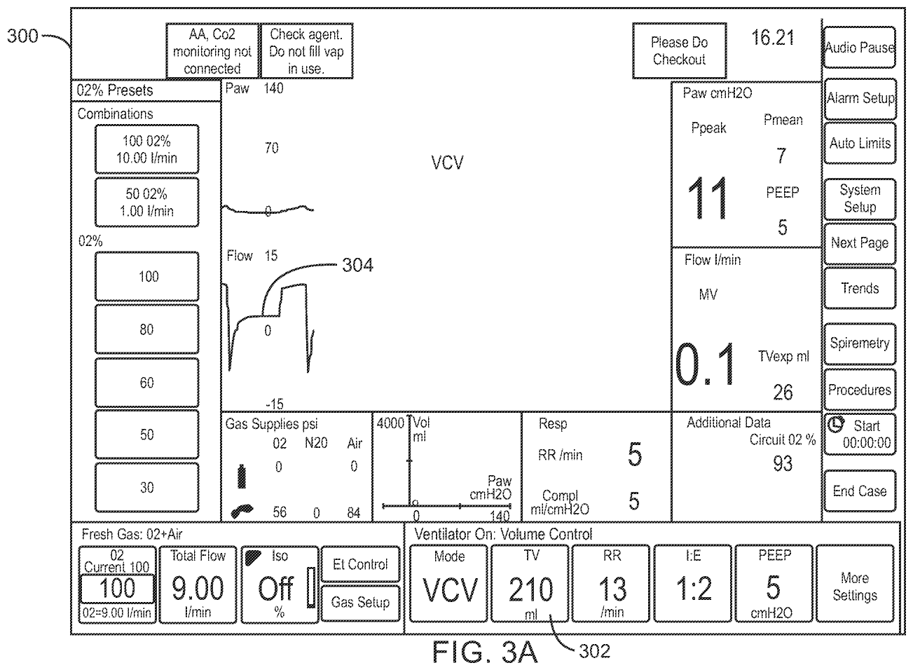

FIG. 3A is an example user interface with a modified setting menu. In some examples, the user interface 300 can provide any number of setting icons 302 or values. The user interface 300 can include any number of physiological data streams 304 such as heart rate, a Paw, a flow rate, and the like. The user interface 300 can also provide information using setting icons 302 and other regions of a display device, wherein the information can include airway pressure, such as airway pressure (Paw) related data, flow related data, alarm information, setup information, spirometry data, and the like.

In response to a selection of an icon of the user interface 300, a modified setting menu can be displayed. FIG. 3B provides an example modified setting menu. The modified setting menu 306 can include a cancel indicator 308 that provides a current value of the setting, an adjusted value 310 of the setting based on any suitable input, and an adjustment widget 312 or icon that enables increasing or decreasing the adjusted value of the setting, among others. In some examples, the cancel indicator 308 can provide the current setting value, information indicating the current setting value is being displayed, and any other suitable information.

In some examples, the adjustment widget 312 or icon for adjusting the value of the setting can be a spinner widget that increases or decreases the adjusted value 310 of the setting in response to a selection of the spinner widget in the up to down direction, a down to up direction, a left to right direction, a right to left direction, along a diagonal direction, or any other suitable swipe or gesture.

In some examples, the adjusted setting value 310 can modify a setting for one or more flow adjustment devices that may include analog and/or digital adjustment dials and/or other user input devices configured to actuate one or more flow control valves. For example, the adjusted setting value 310 can modify an alarm setting, a respiratory parameter or a gas control parameter, among others. In some examples, the gas control parameter can include a gas flow, NO2 flow, an oxygen flow, an air flow, a total flow, an anesthetic agent percentage, a carbon dioxide setting, or an oxygen percentage. The respiratory parameter can include a tidal volume or a respiration rate. In some examples, a system can modify the adjusted setting value 310 in response to a control knob input detected by a control knob electronically coupled to the system. The control knob can be any suitable knob that provides a digital or analog signal.

In some examples, the modified setting menu 306 can include one or more predetermined values for the setting. The predetermined values can include any number of predetermined values that represent quick select icons, wherein the predetermined values are based on historical physiological patient data, or the like.

In some examples, the adjustment widget 312 can change to a different button size and the background color of the setting value can be changed to indicate that the setting value has been modified or adjusted. In some examples, the adjustment widget 312 can modify the adjusted value 310 more quickly or more slowly depending on a period of time the adjustment widget 312 is selected. For example, the adjustment widget 312 can accelerate the increase or decrease of the adjusted value 310 in response to a longer selection of the adjustment widget 312.

In some examples, the modified setting menu 306 is displayed overlaying the setting being modified. In response to any suitable action that closes the modified setting menu 306, the functionality of the modified setting menu 306 can be terminated or ended, and the user interface 300 can be returned to operating functionality. In some examples, the functionality or selections of other areas of the user interface 300 may be disabled as the modified setting menu 306 is displayed.

In some examples, the modified setting menu 308 can be provided with any suitable shape to reduce an amount of information from the user interface 300 that is obscured or hidden. For example, the cancel indicator 308, the adjusted value 310 and the adjustment widget 312 can be arranged in a square, a rectangle, a circle, or the like, to reduce an amount of information that is obscured.

In some examples, the modified setting menu 308 can be used to modify a setting including a tidal volume, a respiratory rate, a minimum respiratory rate, a PEEP value, an I:E value, a Tpause value, or a ventilation pressure such as a Pmax value, a Pinsp value, a Psupport value, a Tinsp value, a rise rate, a PSV rise rate, a trig window, a flow trigger, an end of breath setting, a backup time, or an exit backup. The setting can also include a backup Pinsp, a backup Tinsp, a bias flow, an expiration trigger, an FiO2 value, a flow value, an inspiration pause value, an inspiration trigger value, a minimum rate, a Phigh, a Plimit, a Plow, a Pmax, or a PS rise time, among others.

FIG. 4 is an example diagram of confirmation gestures detectable by the user interface providing the modified setting menu. The confirmation gestures can enable confirming an adjusted setting value from any number of regions or areas of a user interface 400 provided by a display device. For example, the confirmation gestures can include a force applied to a region 402 of the user interface that displays the adjusted setting value, wherein the force is applied for a period of time that exceeds a threshold. The period of time can be any suitable static value or dynamic value that is provided by user input, determined by an application that generates the user interface and the modified setting menu, or otherwise obtained. In some examples, the confirmation gesture can include a force applied to a region 404 of the user interface that excludes both a display of the adjusted setting value and a dead space region proximate the display of the adjusted setting value, wherein the force is applied for a period of time that exceeds a threshold. The dead space region can prevent any unexpected confirmation gesture while a user provides input to the adjusted setting value using an adjustment widget or any other suitable icon or feature. In some examples, the force applied to the region 404 can be any suitable setting icon or value displayed within the user interface 400. The confirmation gesture can also be applied to other regions 406 of the user interface 400 that are not associated with setting values or icons.

In some examples, areas of the user interface outside of the modified setting menu may not accept input or any selection when the modified setting menu is displayed. Additionally, any combination of the confirmation gestures applied to regions 402, 404, and 406 may be simultaneously enabled. In some examples, the confirmation gesture can include a force applied to a region 402 of the user interface that displays the adjusted setting value followed by a release of the force. The confirmation gesture can also include a pushing force applied to a control knob of a system followed by a release of the force applied to the control knob.

It is to be understood that the modified setting menu and the user interface of FIG. 4 are examples and not intended to limit the scope of the present techniques. In some examples, any suitable user interface and any suitable modified setting menu can be implemented with the techniques herein. For example, the user interface can include information related to any number of values for industrial controls, medical devices, mobile devices, or gaming devices, among others. The user interface can provide any number of different values and any number of the different values can be adjusted with the modified setting menu. In some examples, the modified setting menu can include any number of predetermined values that are selectable, an adjustment widget, a present value of a setting, an adjusted value of the setting based on entered adjustments using the adjustment widget, and any other suitable information. The modified setting menu may enable one or more setting to be adjusted before a confirmation gesture is detected or a cancellation gesture is detected.

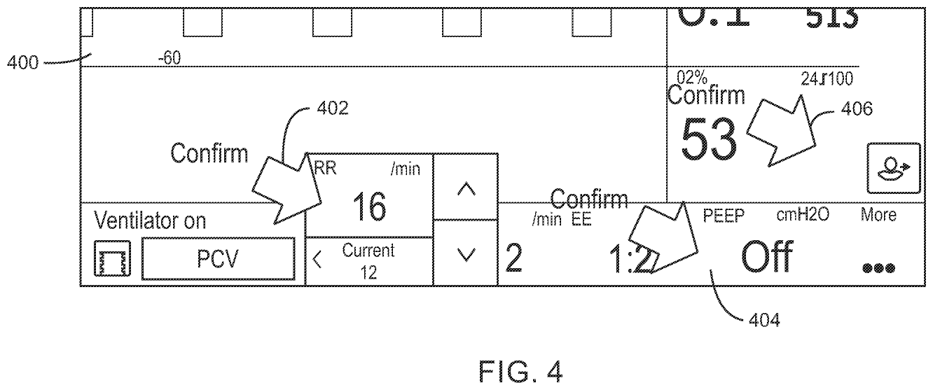

FIG. 5 is an example respiratory or anesthesia device with a control knob and a touchscreen display according to examples herein. Anesthesia machine 500 includes a frame (or housing) 502. In some embodiments, frame 502 may be supported by casters, where the movement of the casters may be controlled (e.g., stopped) by one or more locks. In some examples, the frame 502 may be formed of a plastic material (e.g., polypropylene). In other examples, the frame 502 may be formed of a different type of material (e.g., metal, such as steel).

Anesthesia machine 500 also includes an anesthesia display device 504, a patient monitoring display device 506, a respiratory gas module 508, one or more patient monitoring modules, such as a patient monitoring module 510, a ventilator 512 (explained in more detail below), an anesthetic vaporizer 514, and an anesthetic agent storage bay 516. Anesthesia machine 500 may further include a main power indicator 524, a system activation switch 526 (which, in one example, permits gas flow when activated), an oxygen flush button 528, and an oxygen control 530. Anesthetic vaporizer 514 may vaporize the anesthetic agent and combine the vaporized anesthetic agent with one or more medical grade gases (e.g., oxygen, air, nitrous oxide, or combinations thereof), which may then be delivered to a patient.

Anesthesia machine 500 may additionally include an integrated suction, an auxiliary oxygen flow control, and various other components for providing and/or controlling a flow of the one or more medical grade gases to the patient. For example, anesthesia machine 500 includes one or more pipeline connections 546 to facilitate coupling of the anesthesia machine to pipeline gas sources. Additionally, anesthesia machine 500 includes a cylinder yoke 544, via which one or more gas-holding cylinders 548 may be coupled to the anesthesia machine. Thus, through the pipeline connection and/or cylinder connections, gas may be provided to the anesthesia machine, where the gas may include (but is not limited to) medical air, oxygen, nitrogen, and nitrous oxide. The gas that enters the anesthesia machine may mix with the vaporized anesthetic agent at the anesthetic vaporizer 514, as described above, before being supplied to a patient via the ventilator 512. The anesthesia machine may also include a serial port, a collection bottle connection, a cylinder wrench storage area, and an anesthesia gas scavenging system.

The ventilator 512 may include an expiratory check valve at an expiratory port 520, an expiratory flow sensor at the expiratory port 520, an inspiratory check valve at an inspiratory port 518, an inspiratory flow sensor at the inspiratory port 518, an absorber canister, a manual bag port, a ventilator release, an adjustable pressure-limiting valve, a bag/vent switch, and a bellows assembly. When a patient breathing circuit is coupled to the ventilator 512, breathing gases (e.g., air, oxygen, and/or nitrous oxide mixed with vaporized anesthetic agent) exit the anesthesia machine from the inspiratory port 518 and travel to the patient. Expiratory gases from the patient re-enter the anesthesia machine via the expiratory port 520, where carbon dioxide may be removed from the expiratory gases via the absorber canister.

During operation of the anesthetic vaporizer 514, an operator (e.g., an anesthesiologist) may adjust an amount of vaporized anesthetic agent that is supplied to the patient by adjusting a flow rate of gases from the gas source(s) (e.g., the pipeline gas supply) to the vaporizer. The flow rate of the gases from the gas source to the vaporizer may be adjusted by the operator via adjustment of one or more flow adjustment devices. For example, the flow adjustment devices may include analog and/or digital adjustment dials and/or other user input devices configured to actuate one or more flow control valves of anesthesia machine 500. In some embodiments, a first flow control valve may be positioned between the gas source(s) and the anesthetic vaporizer 514 and may be actuatable via the flow adjustment devices to a fully opened position, a fully closed position, and a plurality of positions between the fully opened position and the fully closed position.

Anesthesia machine 500 may additionally include one or more valves configured to bypass gases from the gas source(s) around the anesthetic vaporizer 514. The valves may enable a first portion of gases to flow directly from the gas source to the inspiratory port 518 and a second portion of gases to flow from the gas source through the anesthetic vaporizer 514 to mix with the vaporized anesthetic agents prior to flowing to the inspiratory port 518. By adjusting a ratio of the first portion of gases relative to the second portion of gases, the operator may control a concentration of vaporized anesthetic agent administered to the patient via the inspiratory port 518.

Further, the adjustments described above may be facilitated at least in part based on output from the respiratory gas module 508. The respiratory gas module 508 may be configured to measure various parameters of the gases exiting the vaporizer and/or being provided to the patient. For example, the respiratory gas module 508 may measure the concentrations of carbon dioxide, nitrous oxide, and the anesthetic agent provided to the patient. Further, the respiratory gas module 508 may measure respiration rate, minimum alveolar concentration, patient oxygen, and/or other parameters. The output from the respiratory gas module 508 may be displayed via a graphical user interface on a display device (e.g., the anesthesia display device 504 and/or the patient monitoring display device 506) and/or used by a controller to provide closed-loop feedback control of the amount of anesthesia provided to the patient.

The ventilator 512 may optionally be coupled to a breathing circuit (not shown) including a plurality of tubes (e.g., gas passages) 522. The breathing circuit may be coupled between an airway of a patient (e.g., via a breathing mask positioned to enclose the mouth and/or nose of the patient or a tracheal intubation tube) and the inspiratory port 518. Gases (e.g., the one or more medical gases, or a mixture of the one or more medical gases and vaporized anesthetic agent from the anesthetic vaporizer 514) may flow from the inspiratory port 518, through the breathing circuit, and into the airway of the patient, where the gases are absorbed by the lungs of the patient. By adjusting the concentration of vaporized anesthetic agent in the gases as described above, the operator may adjust a degree to which the patient is anesthetized.

During conditions in which the breathing circuit is coupled to the airway, the anesthetic agent and/or fresh gas (without the anesthetic agent) may flow into the airway of the patent (e.g., through inhalation) via the inspiratory port 518 and the inspiratory check valve. As an example, the inspiratory check valve may open automatically (e.g., without input or adjustment by the operator) in response to inhalation by the patient and may close automatically in response to exhalation by the patient. Similarly, the expiratory check valve may open automatically in response to exhalation by the patient and may close automatically in response to inhalation by the patient.

In some embodiments, the operator may additionally or alternatively control one or more operating parameters of the anesthesia machine 500 via an electronic controller 540 of the anesthesia machine 500. Controller 540 includes a processor operatively connected to a memory. The memory may be a non-transitory computer-readable medium and may be configured to store computer executable code (e.g., instructions) to be processed by the processor in order to execute one or more routines, such as those described herein. The memory may also be configured to store data received by the processor. Controller 540 may be communicatively coupled (e.g., via wired or wireless connections) to one or more external or remote computing devices, such as a hospital computing system, and may be configured to send and receive various information, such as electronic medical record information, procedure information, and so forth. Controller 540 may also be electronically coupled to various other components of the anesthesia machine 500, such as the anesthetic vaporizer 514, the ventilator 512, the respiratory gas module 508, the anesthesia monitoring display device 504, and the patient monitoring display device 506.

In some examples, the controller 540 can execute instructions that implement the functionality of the display manager 118. For example, the controller 540 can detect a setting selection with a user interface displayed with a touchscreen display device. The display manager 118 can also generate a modified setting menu to be displayed with the user interface, wherein the modified setting menu comprises at least an adjusted setting value and a cancel indicator. The cancel indicator can provide a current setting value for the anesthesia machine 500, such as a respiratory parameter, an alarm setting, or a gas control parameter, among others, along with an indicator of the setting being displayed. The cancel indicator can enable a user of the anesthesia machine 500 to view a current setting value as the modified setting menu is provided or displayed in a region of the user interface that previously provided the setting value. The display manager 118 can also determine whether a confirmation selection confirms the adjusted setting value and, if so, modify the setting based on the confirmation selection. In some examples, the display manager 118 can also determine whether a cancellation selection of the cancel indicator is detected and, if so, close the modified setting menu without adjusting the setting.

In some examples, the controller 540 can detect a physical switch action from any suitable control knob or other switch within or coupled to the anesthesia machine 500. The physical switch action can include input, such as a position change of a control knob, wherein the control knob can include a bag or ventilator switch, an ACGO switch, or an auxiliary gas switch, or an input device, among others. In some examples, the physical switch action from the control knob can be reflected as a setting change in a user interface displayed by the patient monitoring display device 506 or the anesthesia monitoring device 504. For example, the physical switch action can cause the controller 540 to generate the modified setting menu and provide an adjusted setting value. In some examples, the adjustment widget of the modified setting menu can be increased or decreased based on the physical switch action.

In some examples, the range of the adjustment widget is limited based on a minimum value and a maximum value for a setting. For example, the adjustment widget can enable increasing a setting value to the maximum value or decreasing the setting value to the minimum value. The maximum value and the minimum value can be any suitable predetermined values representing a range for a setting. In some examples, audio alerts can be provided as the adjustment widget modifies the setting value within the modified setting menu, as a confirmation gesture is detected to change the setting value, as a cancel indicator is detected, or the like. The audio alerts can also indicate when setting values are adjusted above or below predetermined thresholds.

In some examples, the controller receives signals from the various sensors of the anesthesia machine 500 and employs the various actuators of the anesthesia machine 500 to adjust operation of the anesthesia machine 500 based on the received signals and instructions stored on the memory of the controller. For example, the flow of gases to the inspiratory port 518 may be controlled via an input device (e.g., keyboard, touchscreen, etc.) coupled to the electronic controller of the anesthesia machine 500. The controller 540 may display operating parameters of the anesthesia machine 500 via the anesthesia display device 504 and/or the patient monitoring display device 506. The controller may receive signals (e.g., electrical signals) via the input device and may adjust operating parameters of the anesthesia machine 500 in response (e.g., responsive) to the received signals.

Controller 540 is shown in FIG. 5 for illustrative purposes, and it is to be understood that controller 540 may be located in various locations within, around, and/or remote from anesthesia machine 500. As an example, controller 540 may include multiple devices/modules that may be distributed throughout anesthesia machine 500. As such, controller 540 may include a plurality of controllers at various locations within anesthesia machine 500. As another example, additionally or alternatively, controller 540 may include one or more devices/modules that are external to anesthesia machine 500, located proximate to (e.g., in a same room) or remote from (e.g., a remote server) anesthesia machine 500. In each example, the multiple devices/modules may be communicatively coupled through wired and/or wireless connections.

Anesthetic vaporizers, such as anesthetic vaporizer 514 shown in FIG. 5, may employ any suitable technique to vaporize a liquid anesthetic agent.

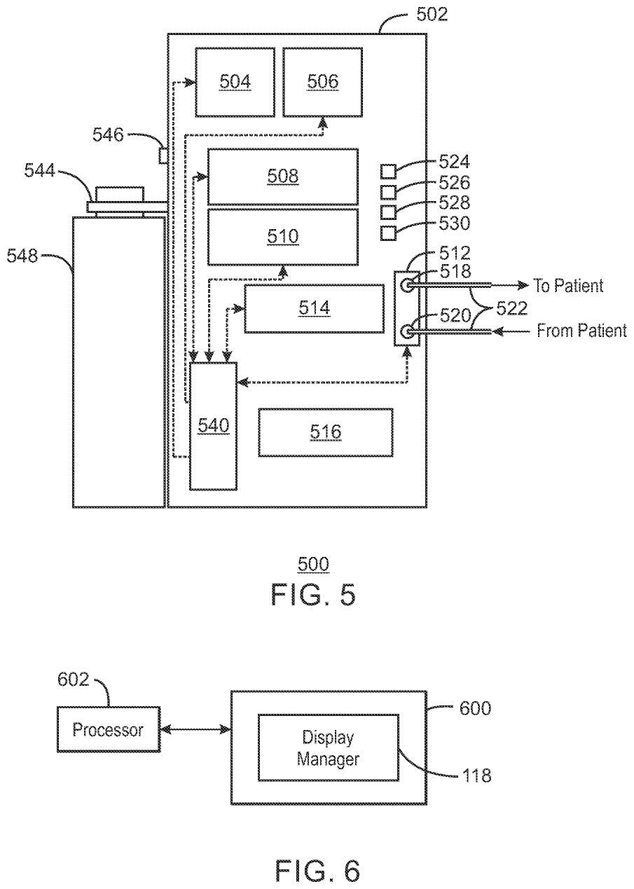

FIG. 6 is an example of a non-transitory machine-readable medium for adjusting a setting using a modified setting menu, in accordance with examples herein. The non-transitory, machine-readable medium 600 can cause a processor 602 to implement the functionalities of method 200. For example, a processor of a computing device (such as processor 102 of FIG. 1), can access the non-transitory, machine-readable media 600.

In some examples, the non-transitory, machine-readable medium 600 can include instructions to execute a display manager 118. For example, the non-transitory, machine-readable medium 600 can include instructions for the display manager 118 that cause the processor 602 to detect a setting selection with a user interface displayed with a touchscreen display device. The display manager 118 can also generate a modified setting menu to be displayed with the user interface, wherein the modified setting menu comprises at least an adjusted setting value and a cancel indicator, and wherein the cancel indicator provides a current setting value. The display manager 118 can also determine whether a confirmation selection confirms the adjusted setting value and, if so, modify the setting based on the confirmation selection. In some examples, the display manager 118 can also determine whether a cancellation selection of the cancel indicator is detected and, if so, close the modified setting menu without adjusting the setting. In some examples, the non-transitory, machine-readable medium 600 can include instructions to implement any combination of the techniques of the method 200 described above.

As used herein, an element or step recited in the singular and proceeded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional such elements not having that property. The terms “including” and “in which” are used as the plain-language equivalents of the respective terms “comprising” and “wherein.” Moreover, the terms “first,” “second,” and “third,” etc. are used merely as labels, and are not intended to impose numerical requirements or a particular positional order on their objects.

Embodiments of the present disclosure shown in the drawings and described above are example embodiments only and are not intended to limit the scope of the appended claims, including any equivalents as included within the scope of the claims. Various modifications are possible and will be readily apparent to the skilled person in the art. It is intended that any combination of non-mutually exclusive features described herein are within the scope of the present invention. That is, features of the described embodiments can be combined with any appropriate aspect described above and optional features of any one aspect can be combined with any other appropriate aspect. Similarly, features set forth in dependent claims can be combined with non-mutually exclusive features of other dependent claims, particularly where the dependent claims depend on the same independent claim. Single claim dependencies may have been used as practice in some jurisdictions require them, but this should not be taken to mean that the features in the dependent claims are mutually exclusive.

1. A system for controlling healthcare settings comprising:
a processor to:
detect a setting selection with a user interface displayed with a touchscreen display device;
generate a modified setting menu to be displayed with the user interface, wherein the modified setting menu comprises at least an adjusted setting value and a cancel indicator, wherein the cancel indicator provides a representation of a current setting value for one or more of the healthcare settings, and wherein the modified setting menu is displayed overlaying a setting being modified;
determine whether a confirmation selection confirms the adjusted setting value and, if so, modify the current setting value to the adjusted setting value based on the confirmation selection; and
determine whether a cancellation selection of the cancel indicator is detected and, if so, close the modified setting menu without adjusting the current setting value.

2. The system of claim 1, wherein the system is a respiratory device or an anesthesia device.

3. The system of claim 1, wherein the setting comprises an alarm setting, a respiratory parameter, or a gas control parameter.

4. The system of claim 3, wherein the gas control parameter comprises a gas flow, NO2 flow, an oxygen flow, an air flow, a total flow, an anesthetic agent percentage, a carbon dioxide setting, or an oxygen percentage.

5. The system of claim 3, wherein the respiratory parameter comprises a tidal volume, a ventilation pressure, or a respiration rate.

6. The system of claim 1, wherein the modified setting menu further comprises an adjustment widget that detects an increased adjustment to the setting or a decreased adjustment to the setting.

7. The system of claim 6, wherein the processor is to modify the adjusted setting value in response to an adjustment selection of the adjustment widget, wherein the adjustment widget is a spinner widget.

8. The system of claim 1, wherein the determining whether the confirmation selection confirms the adjusted setting value comprises detecting a confirmation gesture applied to the user interface.

9. The system of claim 8, wherein the confirmation gesture comprises a force applied to a region of the user interface that displays the adjusted setting value, wherein the force is applied for a period of time that exceeds a threshold.

10. The system of claim 8, wherein the confirmation gesture comprises a force applied to a region of the user interface that displays the adjusted setting value followed by a release of the force or wherein the confirmation gesture comprises a pushing force applied to a control knob of the system followed by a release of the force applied to the control knob.

11. The system of claim 8, wherein the confirmation gesture comprises a force applied to a region of the user interface that excludes both a display of the adjusted setting value and a dead space region proximate the display of the adjusted setting value.

12. The system of claim 1, wherein the modified setting menu comprises one or more predetermined values for the setting and wherein the processor is to determine the one or more predetermined values for the setting based on historical physiological data for a patient.

13. The system of claim 1, wherein the processor is to modify the adjusted setting value in response to a control knob input detected by a control knob electronically coupled to the system.

14. A method for controlling healthcare settings comprising:
detecting a setting selection with a user interface displayed with a touchscreen display device;
generating a modified setting menu to be displayed with the user interface, wherein the modified setting menu comprises at least an adjusted setting value and a cancel indicator, wherein the cancel indicator provides a representation of a current setting value for one or more of the healthcare settings, and wherein the modified setting menu is displayed overlaying a setting being modified;
determining whether a confirmation selection confirms the adjusted setting value and, if so, modifying the current setting value to the adjusted setting value based on the confirmation selection; and
determining whether a cancellation selection of the cancel indicator is detected and, if so, closing the modified setting menu without adjusting the current setting value.

15. The method of claim 14, wherein the setting comprises an alarm setting, a respiratory parameter, or a gas control parameter.

16. The method of claim 14, wherein the modified setting menu further comprises an adjustment widget that detects an increased adjustment to the setting or a decreased adjustment to the setting.

17. The method of claim 16, further comprising modifying the adjusted setting value in response to an adjustment selection of the adjustment widget, wherein the adjustment widget is a spinner widget.

18. The method of claim 14, wherein the determining whether the confirmation selection confirms the adjusted setting value comprises detecting a confirmation gesture applied to the user interface.

19. A non-transitory computer-readable medium comprising a plurality of machine-executable instructions that, in response to execution by a processor, cause the processor to:
detect a setting selection with a user interface displayed with a touchscreen display device;
generate a modified setting menu to be displayed with the user interface, wherein the modified setting menu comprises at least an adjusted setting value and a cancel indicator, wherein the cancel indicator provides a representation of a current setting value for one or more of the healthcare settings, and wherein the modified setting menu is displayed overlaying a setting being modified;
determine whether a confirmation selection confirms the adjusted setting value and, if so, modify the current setting value to the adjusted setting value based on the confirmation selection; and
determine whether a cancellation selection of the cancel indicator is detected and, if so, close the modified setting menu without adjusting the current setting value.

20. The non-transitory computer-readable medium of claim 19, wherein the setting comprises an alarm setting, a respiratory parameter, or a gas control parameter.
